(19) United States
(12) Patent Application Publication
(10) Pub. No.: US 2023/0338712 A1
(43) Pub. Date: Oct. 26, 2023

(54) CATHETER SYSTEM HAVING A GUIDEWIRE AND RELATED METHODS

(71) Applicant: Becton, Dickinson and Company, Franklin Lakes, NJ (US)

(72) Inventors: Jonathan Karl Burkholz, Salt Lake City, UT (US); Arnold Gatoro, Salt Lake City, UT (US); Megan Scherich, Salt Lake City, UT (US); S. Ray Isaacson, Layton, UT (US); Yiping Ma, Layton, UT (US)

(21) Appl. No.: 18/131,761

(22) Filed: Apr. 6, 2023

Related U.S. Application Data
(60) Provisional application No. 63/332,959, filed on Apr. 20, 2022.

(51) Int. Cl. A61M 25/09; A61M 25/06; A61M 39/06

(52) U.S. Cl. CPC A61M 25/09041; A61M 25/0693; A61M 39/0613; A61M 2039/0258

(57) ABSTRACT

A catheter system may include one or more of the following: a catheter adapter, a catheter, a housing, an introducer needle, a tube, a guidewire advancement feature, a pinch member, and a guidewire. A proximal end of the introducer needle may be secured within the housing. The tube may be disposed within the housing and in fluid communication with the proximal end of the introducer needle. The guidewire advancement feature may extend through a slot in the housing and may be movable along the slot. The pinch member may be disposed within the guidewire advancement feature and configured to move along an exterior of the tube with the guidewire advancement feature. The guidewire may be disposed within the tube. In response to moving the guidewire advancement feature distally along the slot, the pinch member may move the hub distally and the guidewire may be advanced distally.

### RELATED APPLICATIONS

This application claims the benefit of U.S. Provisional Patent Application No. 63/332,959, filed Apr. 20, 2022, and entitled CATHETER SYSTEM HAVING A GUIDEWIRE AND RELATED METHODS, which is incorporated herein in its entirety.

### BACKGROUND

Arterial catheterization is a vital procedure that is used ubiquitously in the hospital setting, both in critically injured and perioperative patients. It is estimated that more than eight million arterial catheters are placed yearly in the United States. Arterial catheters can continuously and accurately measure blood pressure as well as heart rate and pulse contour to allow for immediate recognition of aberrant hemodynamic events and initiation of appropriate treatment. They also provide samples for blood gas analysis without the morbidity associated with repeat arterial puncture. However, use of current arterial catheters can result in significant blood leakage, which can endanger a user. Moreover, there is a risk of infection and introduction of microbes into arteries when using current arterial catheters.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices, systems, and methods. In particular, the present disclosure relates to a catheter system having a guidewire, as well as related devices and methods. In some embodiments, the catheter system may include an arterial catheter system configured for insertion into an artery.

In some embodiments, the catheter system may include a catheter adapter, which may include a distal end, a proximal end, and a catheter adapter lumen extending through the distal end of the catheter adapter and the proximal end of the catheter adapter. In some embodiments, the catheter may extend from the distal end of the catheter adapter. In some embodiments, the catheter system may include a housing, which may include a distal end, a proximal end, and a slot disposed between the distal end of the housing and the proximal end of the housing.

In some embodiments, the catheter system may include an introducer needle comprising a sharp distal tip and a proximal end. In some embodiments, the proximal end of the introducer needle may be secured within the housing. In some embodiments, the introducer needle may extend through the catheter. In some embodiments, the catheter system may include a tube disposed within the housing and in fluid communication with the proximal end of the introducer needle. In some embodiments, the catheter system may include a guidewire advancement feature extending through the slot and movable along the slot.

In some embodiments, the catheter system may include one or more pinch members. In some embodiments, the pinch members may be disposed within the guidewire advancement feature and configured to move along an exterior of the tube with the guidewire advancement feature. In some embodiments, the catheter system may include a guidewire, which may include a distal end and a proximal end. In some embodiments, the proximal end of the guidewire may be coupled to a hub. In some embodiments, the guidewire may be disposed within the tube. In some embodiments, in response to moving the guidewire advancement feature distally along the slot, the pinch members may move the hub distally and the guidewire is advanced distally.

In some embodiments, the catheter system may include a flash chamber, which may be disposed at a proximal end of the tube and/or may extend proximal to the housing. In some embodiments, an inner diameter of the flash chamber may be greater than an inner diameter of the tube. In some embodiments, the flash chamber may be non-removably coupled to the proximal end of the housing. In some embodiments, the proximal end of the housing may include a luer adapter. In some embodiments, the flash chamber may be removably coupled to the luer adapter.

In some embodiments, the flash chamber may include a dispenser inserted into the proximal end of the housing and a compressible portion. In some embodiments, in response to removal of the flash chamber from the housing and compression of the compressible portion, the flash chamber may be configured to dispense a drop of blood through the dispenser. In some embodiments, a proximal end of the flash chamber make include an air vent configured to pass air and not blood. In some embodiments, a proximal end of the flash chamber may be closed, and the flash chamber may include an air vent distal to the proximal end of the flash chamber. In some embodiments, the tube may be transparent.

In some embodiments, the introducer needle may include a needle bump. In some embodiments, the catheter system may include a needle safety clip disposed within the catheter adapter. In some embodiments, the introducer needle may be configured to be removed from the catheter and the catheter adapter in response to proximal movement of the housing with respect to the catheter adapter. In some embodiments, in response to removal of the introducer needle from the catheter adapter, the needle bump may catch on the needle safety clip and the needle safety clip shields the sharp distal tip of the introducer needle.

In some embodiments, the catheter system may include a septum disposed within the catheter adapter lumen configured to seal the catheter adapter lumen at arterial blood pressure. In some embodiments, the septum may include a slit therethrough. In some embodiments, there may not be any air vents between an outer surface of the septum and an inner surface of the catheter adapter forming the catheter adapter lumen. In some embodiments, the introducer needle may include a notch. In some embodiments, blood may be configured to flow from inside the introducer needle through the notch and into a space between the introducer needle and the catheter.

In some embodiments, a method of arterial catheterization may include inserting the catheter of the catheter system into an artery. In some embodiments, the method may include advancing the guidewire advancement feature along the slot from a proximal position to a distal position such that the guidewire moves distally beyond the sharp distal tip of the introducer needle. In some embodiments, the catheter system may include the flash chamber, which may be disposed at the proximal end of the tube and/or extend proximal to the housing.

In some embodiments, the flash chamber may include the dispenser inserted into the proximal end of the housing and the compressible portion. In some embodiments, the method may include removing the flash chamber from the housing and compressing the compressible portion such that the flash chamber dispenses a drop of blood through the dispenser. In some embodiments, the method may include moving the housing proximally with respect to the catheter adapter to remove the introducer needle from the catheter and the catheter adapter. In some embodiments, the introducer needle may include a needle bump. In some embodiments, the catheter system may include a needle safety clip disposed within the catheter adapter.

In some embodiments, in response to removal of the introducer needle from the catheter adapter, the needle bump catches on the needle safety clip and the needle safety clip shields the sharp distal tip of the introducer needle. In some embodiments, the housing may be moved proximally with respect to the catheter adapter to remove the introducer needle from the catheter and the catheter adapter when the guidewire is in an advanced position and may extend beyond the sharp distal tip of the introducer needle. In some embodiments, the guidewire may not interfere with shielding of the sharp distal tip by the needle safety clip.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is a side view of an example catheter system, illustrating an example guidewire advancement feature between a distal position and a proximal position, according to some embodiments;

FIG. 1B is a cross-sectional view of the catheter system, illustrating the guidewire advancement feature between the distal position and the proximal position, according to some embodiments;

FIG. 1C is a cross-sectional view of the catheter system, illustrating the guidewire advancement feature in the distal position, according to some embodiments;

FIG. 1D is an enlarged cross-sectional view of a portion of the catheter system, illustrating the guidewire advancement feature between the distal position and the proximal position, according to some embodiments;

FIG. 2A is a cross-sectional view of an example proximal end of the catheter system, according to some embodiments;

FIG. 2B is a cross-sectional view of another example proximal end of the catheter system, according to some embodiments;

FIG. 2C is a cross-sectional view of another example proximal end of the catheter system, according to some embodiments;

FIG. 2D is a cross-sectional view of another example proximal end of the catheter system, according to some embodiments;

FIG. 2E is a cross-sectional view of another example proximal end of the catheter system, according to some embodiments;

FIG. 2F is a cross-sectional view of an example flash chamber having a compressible portion, according to some embodiments;

FIG. 3A is a partial cutaway, upper perspective view of the catheter system, according to some embodiments;

FIG. 3B is a cross-sectional view of another example guidewire advancement feature within the catheter system, according to some embodiments;

FIG. 3C is an upper perspective view of an example piece of the guidewire advancement feature, according to some embodiments;

FIG. 3D is an upper perspective view of another example piece of the guidewire advancement feature configured to couple to the piece of the guidewire advancement feature of FIG. 3C, according to some embodiments;

FIG. 3E is a cross-sectional view of another example guidewire advancement feature within the catheter system, according to some embodiments;

FIG. 3F is another cross-sectional view of the guidewire advancement feature of FIG. 3E within the catheter system, according to some embodiments; and

FIG. 3G is a cross-sectional view of another example guidewire advancement feature within the catheter system, according to some embodiments.

### DESCRIPTION OF EMBODIMENTS

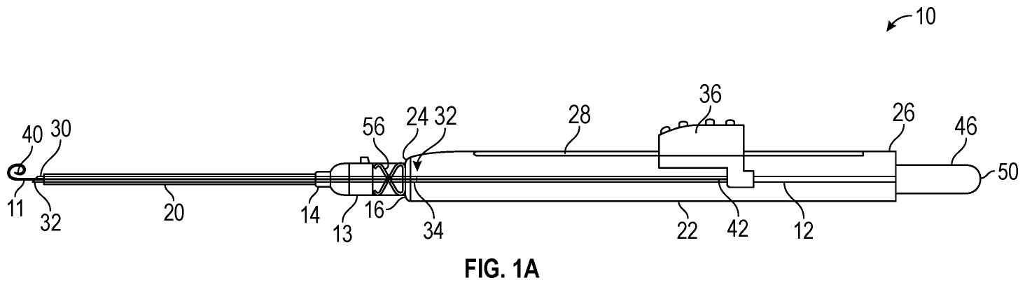

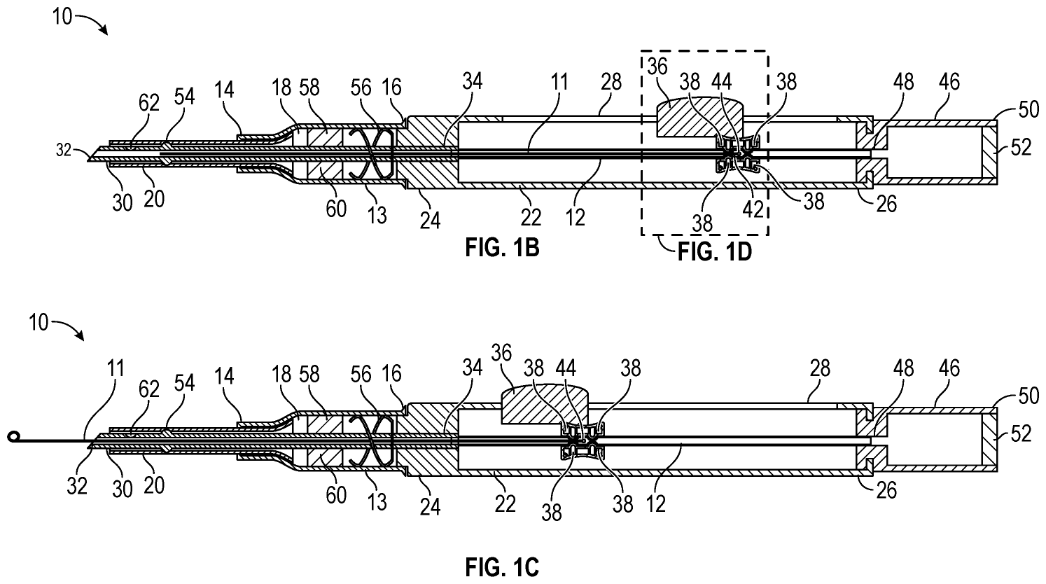

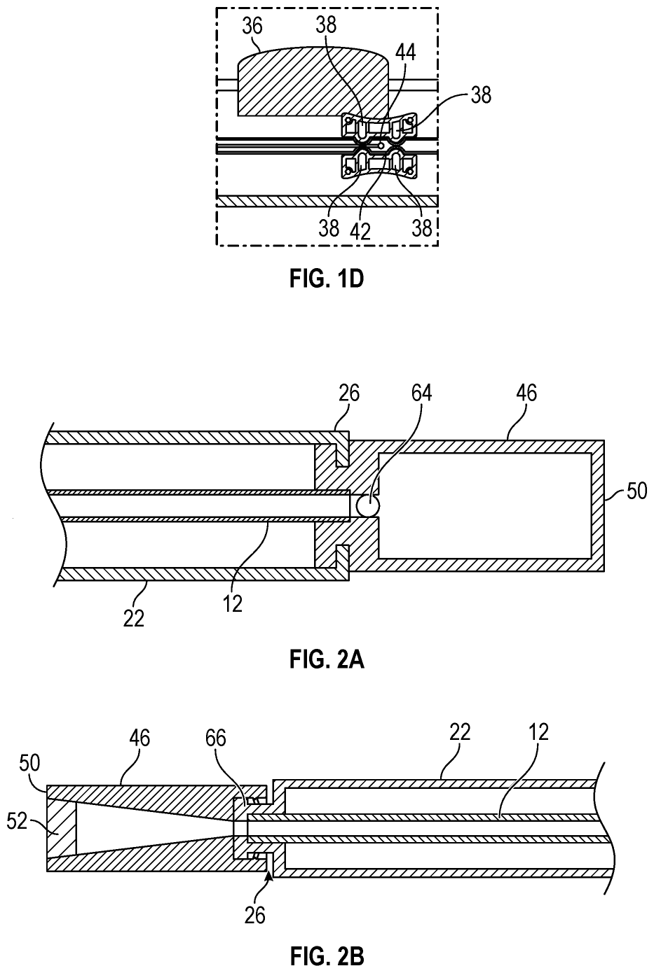

Referring now to FIGS. 1A-1D, a catheter system 10 is illustrated, according to some embodiments. In some embodiments, the catheter system 10 may include an arterial catheter system configured for insertion into an artery. In these embodiments, the catheter system 10 may include significant improvements to existing arterial catheter systems by dramatically reducing blood exposure and infection risk, providing a user with improved artery access confirmation, and improving an overall experience of a patient. Some existing arterial catheter systems, such as, for example, the Teleflex ARROW® Integrated Arterial Catheter, may not provide effective artery access confirmation or blood control, which may result in placement procedures with significant blood exposure risk, infection risk, clean-up costs, and poor patient experience. The Teleflex ARROW® Integrated Arterial Catheter includes a non-rigid, slotted tube out of which significant amounts of blood may leak, endangering the user. As explained in further detail within this disclosure, the catheter system 10 may include one or more of the following, which may provide advantages over the prior art: a vascular access device having a guidewire 11 for arterial placement; a closed system flashback chamber including a tube 12; the guidewire 11 with non-contact advancement through the closed system flashback chamber; a guidewire-compatible introducer needle safety; blood control configured to operate under arterial pressure; and magnetic introducer needle guidance technology.

In some embodiments, the catheter system 10 may include a catheter adapter 13, which may include a distal end 14, a proximal end 16, and a catheter adapter lumen 18 extending through the distal end 14 of the catheter adapter 13 and the proximal end 16 of the catheter adapter 13. In some embodiments, the catheter adapter 13 may be straight, not having a side port and/or and integrated extension tube extending from the side port. In some embodiments, infusion and/or blood draw may occur through the tube 12.

In some embodiments, a catheter 20 may extend from the distal end 14 of the catheter adapter 13. In some embodiments, the catheter system 10 may include the vascular access device, which may include a housing 22. In some embodiments, the housing 22 may include a distal end 24, a proximal end 26, and a slot 28 disposed between the distal end 24 of the housing 22 and the proximal end 26 of the housing 22.

In some embodiments, the catheter system 10 may include an introducer needle 30, which may include a sharp distal tip 32 and a proximal end 34. In some embodiments, the proximal end 34 of the introducer needle 30 may be secured within the housing 22, which may be rigid or semi-rigid. In some embodiments, the introducer needle 30 may extend through the catheter 20. In some embodiments, the catheter system 10 may include the tube 12 disposed within the housing 22 and in fluid communication with the proximal end 34 of the introducer needle 30. In some embodiments, a distal end of the tube 12 may be coupled to the proximal end 34 of the introducer needle 30 such that a fluid tight seal is formed and blood travelling from the introducer needle 30 to the tube 12 may not leak out. In some embodiments, the distal end of the tube 12 may be fitted within, around, and/or bonded with the proximal end 34 of the introducer needle 30. In some embodiments, the proximal end 34 of the introducer needle 30 may be coupled to and secured within the distal end 24 of the housing 22 such that the distal end 24 of the housing 22 acts as a needle hub. In some embodiments, the distal end of the tube 12 may be integrated with and/or sealingly coupled to the distal end 24 of the housing 22. In these and other embodiments, the distal end of the tube 12 may be proximate or spaced apart from the proximal end 34 of the introducer needle 30. In some embodiments, blood may be prevented from flowing in the housing 22 outside of the tube 12 proximal to the distal end of the tube 12, such that blood is prevented from exiting the slot 28.

In some embodiments, the tube 12 may provide a closed-system flashback chamber through which blood may flow. In some embodiments, the tube 12 may be clear or transparent, which may allow the user to visualize entry of blood into the tube 12, signaling placement of the introducer needle 30 in the artery. In some embodiments, the tube 12 may prevent blood from entering a lumen of the housing 22 and/or exiting the slot 28.

In some embodiments, the catheter system 10 may include a guidewire advancement feature 36 extending through the slot 28 and movable along the slot 28. In some embodiments, the guidewire advancement feature 36 may be configured to provide bi-directional movement of the guidewire 11 without direct contact of the guidewire 11. In some embodiments, the catheter system 10 may include one or more pinch members 38 pinching or compressing the tube 12. In some embodiments, the guidewire 11 may be disposed within the tube 12. In some embodiments, the guidewire advancement feature 36 and the pinch members 38 may facilitate advancement and/or retraction of the guidewire 11 without contact of the guidewire by the user or the environment. Thus, in some embodiments, the guidewire 11 in a closed system may decrease a risk of contamination of the guidewire 11 and entry of bacteria into the artery. In some embodiments, in response to moving the guidewire advancement feature 36 distally along the slot 28, the pinch members 38 may move the hub 44 distally and the guidewire 11 may be advanced distally in response. In some embodiments, the guidewire advancement feature 36 may be configured for sliding by a hand of the user, and an upper portion of the guidewire advancement feature 36 may include a tab configured to contact a finger of the user. In some embodiments, the tab may include one or more protrusions to facilitate contact with the finger.

As illustrated in FIGS. 1B-1D, in some embodiments, the pinch members 38 may include molded bumps. In some embodiments, the pinch members 38 may be monolithically formed as a single unit with the guidewire advancement feature 36. In some embodiments, the pinch members 38 may be disposed within the guidewire advancement feature 36 and configured to move along an exterior of the tube 12 with the guidewire advancement feature 36. In some embodiments, the guidewire 11 may include a distal end 40 and a proximal end 42. In some embodiments, the distal end 40 may be blunt or include an otherwise atraumatic tip. In some embodiments, the guidewire 11 may be constructed of metal or another suitable material. In some embodiments, the guidewire 11 may navigate an artery and may act as a guide the catheter 20 can rapidly follow for easier delivery into the artery.

In some embodiments, the proximal end of the guidewire 11 may be coupled to a hub 44, which may have a greater outer diameter than the guidewire 11 to facilitate movement by the pinch members 38. In some embodiments, the hub 44 may be wedge-shaped or another suitable shape. In some embodiments, the proximal end of the guidewire 11 may be looped or otherwise bent to form the hub 44.

In some embodiments, the catheter system 10 may include a flash chamber 46, which may be disposed at a proximal end 48 of the tube 12 and/or extend proximal to the housing 22 to facilitate visualization. In some embodiments, an inner diameter of the flash chamber 46 may be greater than an inner diameter of the tube 12 to facilitate collection of blood. As illustrated in FIGS. 1B-1C, in some embodiments, the flash chamber 46 may be non-removably coupled to the proximal end 26 of the housing 22. In some embodiments, a proximal end 50 of the flash chamber 46 make include an air vent 52 configured to pass air and not blood. In these and other embodiments, the air vent 52 may allow blood to fill the entire flash chamber 46 while maintaining complete blood containment.

In some embodiments, the housing 22 may include one or more vents to maintain atmospheric pressure around the tube 12 and allow movement of air within a lumen of the housing 22 during translation in a distal direction and/or a proximal direction of the guidewire advancement feature 36 while facilitating aseptic delivery of the guidewire 11. In some embodiments, the distal end of the tube 12 may be bonded to the housing 22, which may secure the tube 12 within the housing 22. In some embodiments, the proximal end 48 of the tube 12 may be bonded to the housing 22 and/or the flash chamber 46, which may secure the tube 12. In some embodiments, the distal end of the tube 12 and the proximal end of the tube 12 may be vented to atmosphere and in fluid communication with the introducer needle 30.

In some embodiments, the introducer needle 30 may include a needle bump 54. In some embodiments, the catheter system 10 may include a needle safety clip 56 (or another type of passive needle safety configured to shield the sharp distal tip 32) disposed within the catheter adapter 13. In some embodiments, the introducer needle 30 may be configured to be removed from the catheter 20 and the catheter adapter 13 in response to proximal movement of the housing 22 with respect to the catheter adapter 13. In some embodiments, in response to removal or proximal withdrawal of the introducer needle 30 from the catheter adapter 13, the needle bump 54 may catch on the needle safety clip 56 and release the needle safety clip 56 from the catheter adapter 13. In some embodiments, the needle safety clip 56 may shield the sharp distal tip 32 of the introducer needle 30 upon further proximal withdrawal for the introducer needle 30. One example of the needle safety clip 56 is described in U.S. Pat. No. 8,647,313, filed Oct. 22, 2012, entitled “SPRING CLIP SAFETY IV CATHETER,” wherein is hereby incorporated by reference in its entirety.

In some embodiments, the catheter system 10 may include a septum 58 disposed within the catheter adapter lumen 18 and configured to seal the catheter adapter lumen 18 at arterial blood pressure. In further detail, in some embodiments, the septum 58 may be configured to seal the catheter adapter lumen 18 at systolic blood pressures of 80 to 120 mmHg. In some embodiments, a number and/or size of air vents between an outer surface of the septum 58 and an inner surface of the catheter adapter 13 may be decreased compared to a venous system due to arterial blood pressure. For example, there may be 1, 2, or 3 air vents between the outer surface of the septum 58 and the inner surface of the catheter adapter 13. In some embodiments, the air vents may be configured to pass air but not blood. In some embodiments, there may not be any air vents between the outer surface of the septum 58 and the inner surface of the catheter adapter 13 forming the catheter adapter lumen 18. In these and other embodiments, the septum 58 may include a slit 60 therethrough, and the arterial blood pressure may be strong enough to force air through the slit 60 to provide venting. In some embodiments, a diameter of the slit 60 may be configured to allow air to pass in response to arterial blood pressure but not blood. In some embodiments, the arterial blood pressure may be strong enough to force air between an outer surface of the introducer needle 30 and an inner surface of the septum 58. In these and other embodiments, the septum 58 may not include a slit 60.

In some embodiments, the introducer needle 30 may include a notch 62, which may be disposed within the catheter 20. In some embodiments, blood may be configured to flow from inside the introducer needle 30 through the notch 62 and into a space between the introducer needle 30 and the catheter 20. Thus, the catheter system 10 may provide immediate visual confirmation of arterial access to the user.

In some embodiments, a method of arterial catheterization may include inserting the catheter 20 of the catheter system 10 into an artery. In some embodiments, the method may include advancing the guidewire advancement feature 36 along the slot 28 from a proximal position to a distal position, illustrated, for example, in FIG. 1C, such that the guidewire 11 moves distally beyond the sharp distal tip 32 of the introducer needle 30. In some embodiments, when the guidewire advancement feature 36 is in the proximal position, the distal end 40 of the guidewire 11 may be proximal to a distal tip of the catheter 20. In some embodiments, in response to removal of the introducer needle 30 from the catheter adapter 13, the needle bump 54 may catch on the needle safety clip 56 and the needle safety clip 56 may shield the sharp distal tip 32 of the introducer needle 30. In some embodiments, when the guidewire 11 is in an advanced position and extends beyond the sharp distal tip 32 of the introducer needle 30, the housing 22 may be moved proximally with respect to the catheter adapter 13 to remove the introducer needle 30 from the catheter 20 and the catheter adapter 13. In some embodiments, the needle safety clip 56 may be configured such that the guidewire 11 may not interfere with shielding of the sharp distal tip 32 by the needle safety clip 56.

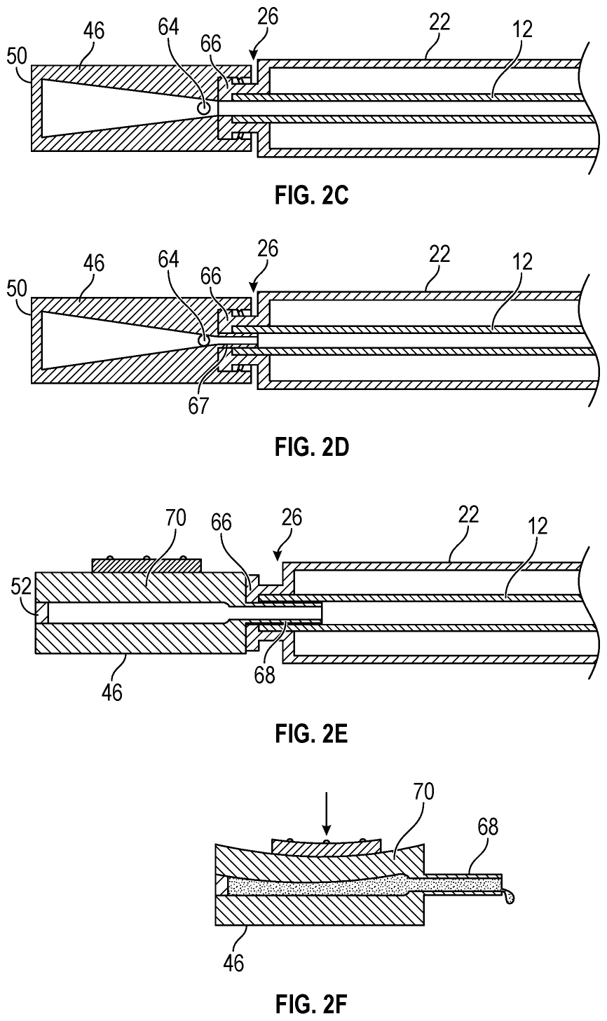

Referring now to FIG. 2A, in some embodiments, the proximal end 50 of the flash chamber 46 may be closed, and the flash chamber 46 may include an air vent 64 distal to the proximal end 50 of the flash chamber 46. In some embodiments, the air vent 64 may be configured to pass air and not blood. In some embodiments, the air vent 64 may be disposed at a distal end of the flash chamber 46, which may maintain some unvented air in the flash chamber 46 to provide arterial pulse visualization, thereby providing sustained visual confirmation of proper arterial access. In some embodiments, the air vent 64 may be disposed at the distal end of the flash chamber 46 within a narrowed passage adjacent to the proximal end of the tube 12, as illustrated, for example, in FIG. 2A. In some embodiments, all or a portion of the flash chamber 46 may be transparent, which may allow the user to visualize the arterial pulse. In some embodiments, an inner wall of the flash chamber 46 may include a curvature for magnification to facilitate visualization of the arterial pulse by the user.

Referring now to FIGS. 2B-2D, in some embodiments, the proximal end 50 of the housing 22 may include a luer adapter 66, such as, for example, a female luer adapter. In some embodiments, the flash chamber 46 may be removably coupled to the luer adapter 66. In some embodiments, the flash chamber 46 may include an annular collar, which may include threading configured to couple to the luer adapter 66. As illustrated in FIG. 2D, in some embodiments, the flash chamber 46 may include a male luer 67 configured to insert into the luer adapter 66, similar to the dispenser 68 illustrated in FIG. 2D. In some embodiments, the male luer 67 may facilitate formation of a fluid seal with the luer adapter 66. In any of the embodiments of FIGS. 1-3, the introducer needle 30 may be removed after distal advancement of the guidewire 11 and successful threading of the catheter 20 into the artery. In some embodiments, the housing 22 may include a visual mark that indicates the distal end 40 of the guidewire 11 is at or aligned with the sharp distal tip 32 of the introducer needle 30 during a process of advancing the guidewire 11.

Referring now to FIGS. 2E-2F, in some embodiments, the flash chamber 46 may include a dispenser 68 inserted into the proximal end 26 of the housing 22. In some embodiments, the flash chamber 46 may include a compressible portion 70. In some embodiments, in response to removal of the flash chamber 46 from the housing 22 and compression of the compressible portion 70, the flash chamber 46 may be configured to dispense a drop of blood through the dispenser 68. Thus, in some embodiments, the flash chamber 46 may be configured for acquiring a point-of-care sample for dispending on a point of care test cartridge for point of care blood testing.

In some embodiments, the flash chamber 46 may be removed from the housing 22 and the compressible portion 70 may be compressed such that the flash chamber 46 dispenses a drop of blood through the dispenser 68. In some embodiments, the housing 22 may be moved proximally with respect to the catheter adapter 13 to remove the introducer needle 30 from the catheter 20 and the catheter adapter 13.

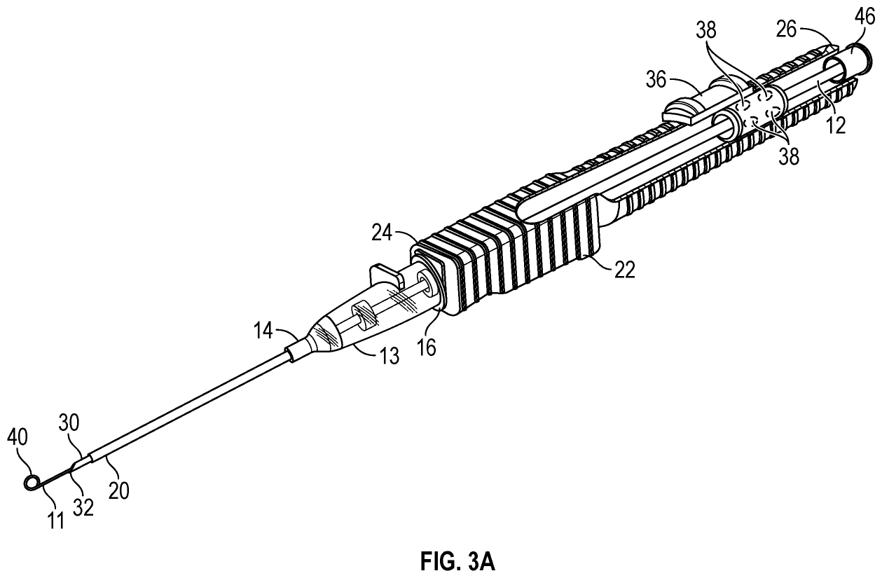

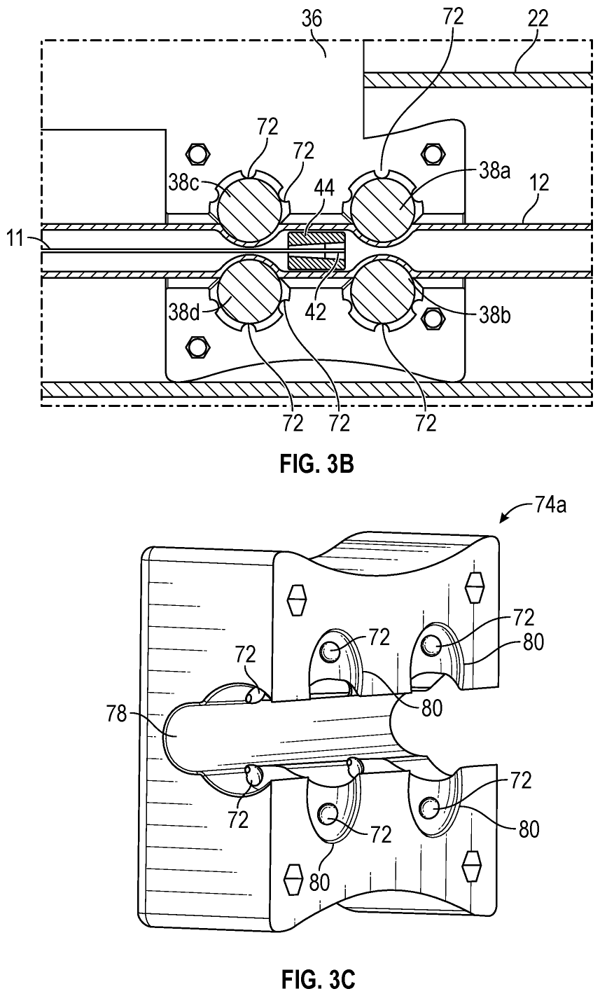

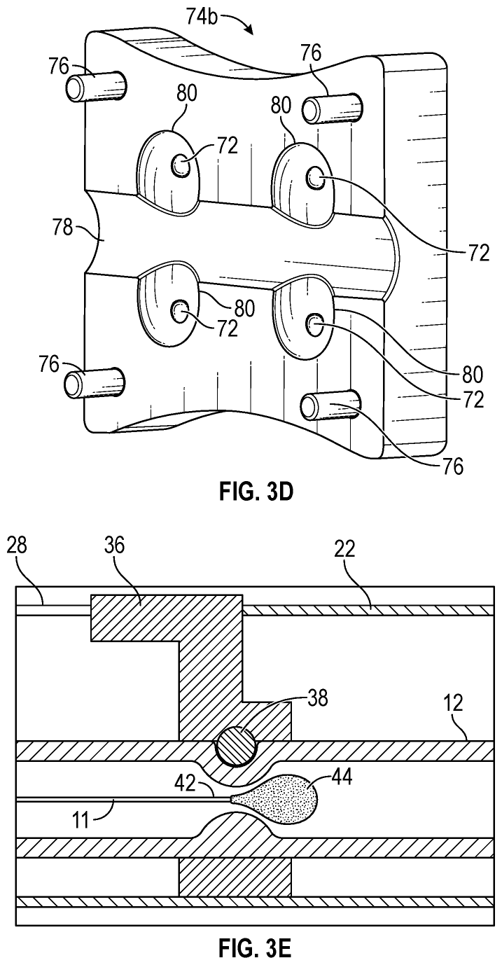

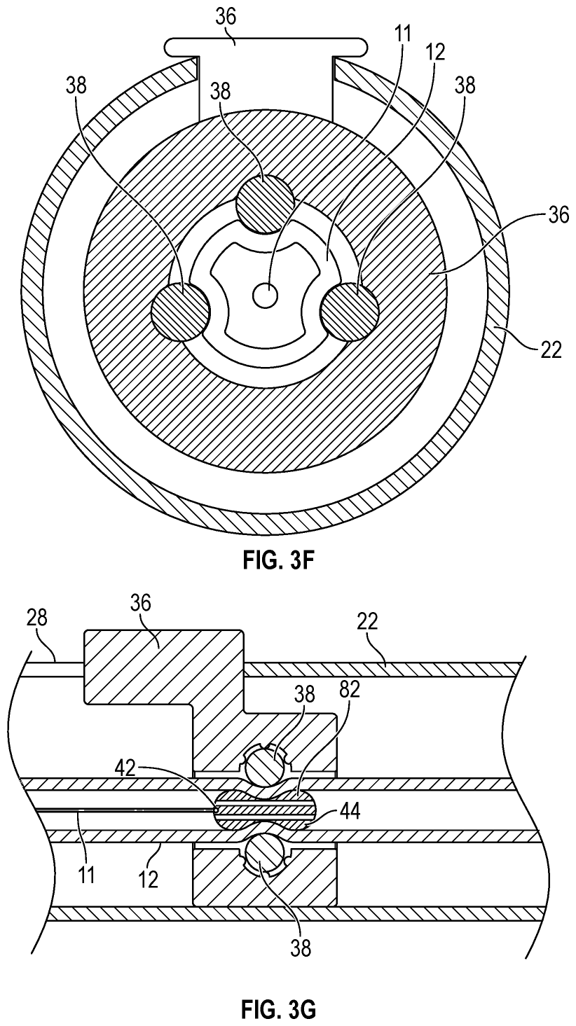

Referring now to FIGS. 3A-3D, in some embodiments, the guidewire advancement feature 36 may include the hub 44 disposed within the guidewire advancement feature 36 and a lumen of the tube 12. As illustrated in FIG. 3B, in some embodiments, the guidewire advancement feature 36 may include the pinch members 38, which may include a pair of opposing pinch members 38a,b configured to pinch the tube 12. In some embodiments, the pair of opposing pinch members 38a,b may be disposed within the guidewire advancement feature 36 proximal to the hub 44 and configured to move along the tube 12 with the guidewire advancement feature 36. In some embodiments, the tube 12 may include an extension tube, which may be flexible to facilitate pinching or compression of the extension tube. In other embodiments, blood may be configured to flow through the lumen of the tube 12 and/or around the hub 44.

In some embodiments, the guidewire 11 may extend distally from the hub 44. In some embodiments, the guidewire 11 may be disposed within the lumen of the tube 12. In some embodiments, in response to moving the guidewire advancement feature 36 distally along the tube 12, the pair of opposing pinch members 38a,b may push the hub 44 distally, and the guidewire 11 may be advanced distally.

In some embodiments, the pinch members 38 may include another pair of opposing pinch members 38c,d configured to pinch the tube 12. In some embodiments, the other pair of opposing pinch members 38c,d may be disposed distal to the hub 44 and configured to move along the tube 12 with the guidewire advancement feature 36. In some embodiments, in response to moving the guidewire advancement feature 36 proximally along the tube 12, the pair of opposing pinch members 38c,d may push the hub 44 proximally and the guidewire 11 may be retracted proximally.

In some embodiments, in response to movement of the guidewire advancement feature 36 along the tube 12, the pinch members 38 may rotate with respect to the guidewire advancement feature 36 and the tube 12. In some embodiments, an inner surface of the guidewire advancement feature 36 may include one or more bumps 72 in contact with the pinch members 38, which may reduce friction as the pinch members 38 rotate. In some embodiments, the hub 44 and/or the pinch members 38 may be lubricated with a lubricant, which may reduce friction. In some embodiments, the hub 44 may be constructed of a lubricious material to facilitate movement of the hub 44 within the lumen of the tube 12.

In some embodiments, the guidewire advancement feature 36 may include multiple pieces 74, which may be coupled together via pegs 76 or any other suitable mechanism. In other embodiments, the guidewire advancement feature 36 may be monolithically formed as a single unit. In some embodiments, the guidewire advancement feature 36 may include an aperture 78 extending therethrough and configured to receive the tube 12.

In some embodiments, the guidewire advancement feature 36 may include multiple cutouts 80, which may include the bumps 72. In these and other embodiments, the pinch members 38 may include the spherical balls. In some embodiments, the cutouts 80 may be generally spherical and/or may extend outwardly from the aperture 78. In some embodiments, halves of the cutouts 80 illustrated in the multiple pieces 74 may be joined together to form the cutouts 80.

In some embodiments, the pinch members 38 may be constructed of plastic, metal, or another suitable material. In some embodiments, the pinch members 38 may include spherical balls, ball bearings, wheels, or cylinders, which may be configured to rotate with respect to the guidewire advancement feature 36. In some embodiments, the pinch members 38 may include the wheels, which may be smooth or include feet along their edges. In these embodiments, lubricant may be applied to axels of the wheels to reduce friction.

In some embodiments, the guidewire advancement feature 36 may include one or more textured surfaces to facilitate gripping by the user. In some embodiments, the guidewire advancement feature 36 may be rigid or semi-rigid to facilitate gripping and/or one-handed advancement by the user. In some embodiments, the guidewire advancement feature 36 and the pinch members 38 may facilitate the user advancing and/or retracting the guidewire 11 without contacting the guidewire 11, thereby decreasing a risk of contamination and/or infection.

In some embodiments, the guidewire advancement feature 36 may include any suitable pinch member configured to pinch the tube 12 and facilitate the user advancing and/or retracting the guidewire 11 without contacting the guidewire 11. Other examples of suitable pinch members and/or guidewire advancement features may be further described in U.S. patent application Ser. No. 17,127,588, filed Dec. 18, 2020, entitled “Flush Instrument with Blood Exposure Protection and Related Methods” and U.S. patent application Ser. No. 17/127,623, filed Dec. 18, 2020, entitled “Multi-Lumen Extension Set,” which are hereby incorporated by reference in their entirety.

In some embodiments, the pinch members 38 may be fixed with respect to the guidewire advancement feature 36. In some embodiments, the pinch members 38 may be molded into the guidewire advancement feature 36, such as, for example, illustrated in FIG. 1D. In some embodiments, a number of the pinch members 38 may vary based, for example, on a shape of the hub 44. In some embodiments, the guidewire advancement feature 36 may include the pair of opposing pinch members 38a,b and the other pair of opposing pinch members 38c,d in response to the shape of the hub 44 being cylindrical, for example.

Referring now to FIG. 3E-3F, in some embodiments, the guidewire advancement feature 36 may include three of the pinch members 38. In some embodiments, the pinch members 38 may be evenly spaced around a circumference of the tube 12.

Referring now to FIG. 3G, in some embodiments, the guidewire advancement feature 36 may include a single pair of the pinch members 38. In some embodiments, the hub 44 may include a dog bone shape, and the single pair may be disposed in a middle or depression of the dog bone shape. In some embodiments, blood and/or infusion fluid may be configured to flow through the tube 12, passing the hub 44. In some embodiments, the hub 44 may include one or more channels therethrough to facilitate fluid flow therethrough.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A catheter system, comprising:
a catheter adapter, comprising a distal end, a proximal end, and a catheter adapter lumen extending through the distal end of the catheter adapter and the proximal end of the catheter adapter;
a catheter extending from the distal end of the catheter adapter;
a housing, comprising a distal end, a proximal end, and a slot disposed between the distal end of the housing and the proximal end of the housing;
an introducer needle comprising a sharp distal tip and a proximal end, wherein the proximal end of the introducer needle is secured within the housing, wherein the introducer needle extends through the catheter;
a tube disposed within the housing and in fluid communication with the proximal end of the introducer needle;
a guidewire advancement feature extending through the slot and movable along the slot;
a plurality of pinch members, wherein the plurality of pinch members is disposed within the guidewire advancement feature and configured to move along an exterior of the tube with the guidewire advancement feature; and
a guidewire comprising a distal end and a proximal end, wherein the proximal end of the guidewire is coupled to a hub, wherein the guidewire is disposed within the tube, wherein in response to moving the guidewire advancement feature distally along the slot, the plurality of pinch members moves the hub distally and the guidewire is advanced distally.

2. The catheter system of claim 1, further comprising a flash chamber disposed at a proximal end of the tube and extending proximal to the housing.

3. The catheter system of claim 2, wherein an inner diameter of the flash chamber is greater than an inner diameter of the tube.

4. The catheter system of claim 2, wherein the flash chamber is non-removably coupled to the proximal end of the housing.

5. The catheter system of claim 2, wherein the proximal end of the housing comprises a luer adapter, wherein the flash chamber is removably coupled to the luer adapter.

6. The catheter system of claim 2, wherein the flash chamber comprises a dispenser inserted into the proximal end of the housing and a compressible portion, wherein in response to removal of the flash chamber from the housing and compression of the compressible portion, the flash chamber is configured to dispense a drop of blood through the dispenser.

7. The catheter system of claim 2, wherein a proximal end of the flash chamber comprises an air vent configured to pass air and not blood.

8. The catheter system of claim 2, wherein a proximal end of the flash chamber is closed, wherein the flash chamber comprises an air vent distal to the proximal end of the flash chamber.

9. The catheter system of claim 1, wherein the tube is transparent.

10. The catheter system of claim 1, wherein the introducer needle comprises a needle bump, further comprising a needle safety clip disposed within the catheter adapter, wherein the introducer needle is configured to be removed from the catheter and the catheter adapter in response to proximal movement of the housing with respect to the catheter adapter, wherein in response to removal of the introducer needle from the catheter adapter, the needle bump catches on the needle safety clip and the needle safety clip shields the sharp distal tip of the introducer needle.

11. The catheter system of claim 1, further comprising a septum disposed within the catheter adapter lumen configured to seal the catheter adapter lumen at arterial blood pressure.

12. The catheter system of claim 11, wherein the septum comprises a slit therethrough, wherein there are no air vents between an outer surface of the septum and an inner surface of the catheter adapter forming the catheter adapter lumen.

13. The catheter system of claim 1, wherein the introducer needle comprises a notch, wherein blood is configured to flow from inside the introducer needle through the notch and into a space between the introducer needle and the catheter.

14. A method of arterial catheterization, comprising:
inserting a catheter of a catheter system into an artery, wherein the catheter system comprises:
a catheter adapter, comprising a distal end, a proximal end, and a catheter adapter lumen extending through the distal end of the catheter adapter and the proximal end of the catheter adapter;
the catheter extending from the distal end of the catheter adapter;
a housing, comprising a distal end, a proximal end, and a slot disposed between the distal end of the housing and the proximal end of the housing;
an introducer needle comprising a sharp distal tip and a proximal end, wherein the proximal end of the introducer needle is secured within the housing, wherein the introducer needle extends through the catheter;
a tube disposed within the housing and in fluid communication with the proximal end of the introducer needle;
a guidewire advancement feature extending through the slot and movable along the slot;
a plurality of pinch members, wherein the plurality of pinch members is disposed within the guidewire advancement feature and configured to move along an exterior of the tube with the guidewire advancement feature; and
a guidewire comprising a distal end and a proximal end, wherein the proximal end of the guidewire is coupled to a hub, wherein the guidewire is disposed within the tube, wherein in response to moving the guidewire advancement feature distally along the slot, the plurality of pinch members moves the hub distally and the guidewire is advanced distally; and
advancing the guidewire advancement feature along the slot from a proximal position to a distal position such that the guidewire moves distally beyond the sharp distal tip of the introducer needle.

15. The method of claim 14, further comprising a flash chamber disposed at a proximal end of the tube and extending proximal to the housing.

16. The method of claim 15, wherein the flash chamber comprises a dispenser inserted into the proximal end of the housing and a compressible portion, further comprising removing the flash chamber from the housing and compressing the compressible portion such that the flash chamber dispenses a drop of blood through the dispenser.

17. The method of claim 14, further comprising moving the housing proximally with respect to the catheter adapter to remove the introducer needle from the catheter and the catheter adapter.

18. The method of claim 17, wherein the introducer needle comprises a needle bump, wherein the catheter system further comprises a needle safety clip disposed within the catheter adapter, wherein in response to removal of the introducer needle from the catheter adapter, the needle bump catches on the needle safety clip and the needle safety clip shields the sharp distal tip of the introducer needle.

19. The method of claim 18, wherein the housing is moved proximally with respect to the catheter adapter to remove the introducer needle from the catheter and the catheter adapter when the guidewire is in an advanced position and extends beyond the sharp distal tip of the introducer needle.

20. The method of claim 19, wherein the guidewire does not interfere with shielding of the sharp distal tip by the needle safety clip.
